(19) United States
(12) Patent Application Publication
(10) Pub. No.: US 2026/0004445 A1
(43) Pub. Date: Jan. 1, 2026

(54) MATERIAL INSPECTION SYSTEM

(71) Applicant: FENG CHIA UNIVERSITY, Taichung City (TW)

(72) Inventors: Sze Teng LIONG, Taichung City (TW); Yee Siang GAN, Taichung City (TW); Yi Liang WU, Taichung City (TW); Tsung Yi CHEN, Taichung City (TW)

(21) Appl. No.: 19/233,305

(22) Filed: Jun. 10, 2025

(30) Foreign Application Priority Data
Jun. 26, 2024 (TW) 113123892

(51) Int. Cl. G06T 7/62; G01F 17/00; G01N 21/88; G01N 33/02; G06T 7/277

(52) U.S. Cl. CPC G06T 7/62; G01F 17/00; G01N 21/8851; G06T 7/277; G01N 2021/8874; G01N 33/025; G06T 2207/30188

(57) ABSTRACT

A material inspection system includes a supporting structure, a conveying portion, an image-taking portion, a driving portion, and a volume calculation module. The conveying portion is provided on the supporting structure and can convey a material from a first end to a second end of the conveying portion. The image-taking portion has an imaging lens whose field of view encompasses at least a portion of the moving path of the material on the conveying portion. The driving portion is located on the conveying portion and can drive the material into rotation so that the image-taking portion can take a plurality of depth images of different portions of the material while the material is in rotation. The volume calculation module extracts the depth information in each depth image and feeds the depth information into a deep learning model to calculate the volume of the material.

### FIELD OF THE INVENTION

The present invention relates to image processing technology and more particularly to a material inspection system.

### DESCRIPTION OF THE RELATED ART

One conventional method for grading farm produce is to allow the produce to fall through holes of different fixed sizes. This method, however, tends to result in inaccurate grading, especially when the produce being graded has irregular shapes. If this is the only method that can be used, grading efficiency will be relatively low. One existing solution for the grading of tea leaves, for example, is to use a fresh tea leaf grading machine and a variable-gap grading machine. These machines provide improvement to a certain extent but still have their limitations. The fresh tea leaf grading machine works only by means of the fixed-sized holes and does not allow flexible adjustment. The variable-gap grading machine provides adjustable grading plates but requires downtime for operating the grading plates.

In view of the aforesaid problems, and with the advancement of technology, there have been improvements in techniques for classifying objects according to their volumes. For instance, China Patent No. CN208200270U, “AUTOMATED GUIDED VEHICLE (AGV) CAPABLE OF VOLUME CALCULATION BASED ON 3D CAMERA,” essentially discloses: using a three-dimensional (3D) camera to capture and collect snapshots of goods; obtaining the length, width, and height of the goods by constructing a 3D model; and determining the volume of the goods according to the length, width, and height obtained.

The time required for constructing a 3D model depends on various factors, including the complexity of the model, the required precision, and the tool, as well as the level of the technology, being used, among others. If the objects for which the 3D model is intended have a simple geometric structure or if an existing 3D model library is used, model construction may take only several hours or an even shorter time. However, if the objects have a complicated geometric structure or if a highly detailed 3D model is needed, a longer time lasting several weeks or several months may be required.

Furthermore, regarding volume calculation, the goods in the afore-cited patent have a relatively simple geometric structure, e.g., a cube or cuboid-shaped structure; therefore, only the length, width, and height of the goods have to be obtained to calculate the volume of the goods. If the goods have a complicated geometric structure instead, it will be difficult to precisely calculate the volume using only such dimensions as length, width, and height.

### SUMMARY OF THE INVENTION

The primary objective of the present invention is to provide a material inspection system that dispenses with the conventional step of 3D model construction and that directly extracts depth information and feeds the depth information into a deep learning model in order to calculate the volume of a material. Compared with the prior art, the system shortens the time required for computation and reduces the use of computation resources.

Another objective of the present invention is to provide a material inspection system that can calculate the volume of a complicated geometric structure and that is particularly suitable for materials having an irregular shape and uneven surface.

To achieve the foregoing objectives, the material inspection system provided by the present invention essentially includes a supporting structure, a conveying portion, an image-taking portion, a driving portion, and a volume calculation module. The conveying portion is provided on the supporting structure, has a first end and a second end, and is configured to convey a material from the first end to the second end. The image-taking portion has an imaging lens, and the field of view of the imaging lens encompasses at least a portion of the path along which the material is moved on the conveying portion so that the image-taking portion can take images of the material. The driving portion is located on the conveying portion and is configured to drive the material into rotation such that the portion of the material that faces the image-taking portion is changed at different time points, thereby allowing the image-taking portion to take a plurality of depth images of the different portions of the material while the material is in rotation. The volume calculation module extracts the depth information in each depth image and feeds each piece of depth information into a deep learning model to calculate the volume of the material.

In one embodiment, the material inspection system further includes an object tracking module. The object tracking module identifies the material in each image taken by the image-taking portion, obtains position information of the material through analysis, and marks the position where the material is identified in each image with a tracking label.

In one embodiment, the volume calculation module further analyzes each piece of depth information to produce the corresponding depth matrix, multiplies each depth matrix by the position information pixel-wise to obtain a plurality of corresponding depth values, and determines a material position and a non-material position in each piece of depth information according to the corresponding depth values. The volume calculation module then excludes the portion of each piece of depth information that is determined to be a non-material position, and performs volume calculation based on the portion of each piece of depth information that is determined to be a material position.

In one embodiment, each depth value is 0 or 1. The depth value 1 indicates that the corresponding portion of a piece of depth information is a material position, and the depth value 0 indicates that the corresponding portion of a piece of depth information is a non-material position.

In one embodiment, there are a plurality of such materials, and the object tracking module can track those materials individually at the same time.

In one embodiment, the position information is obtained by determining a centroid according to the following equation, and the centroid is used as a basis for tracking:

$${centroid}_{( x , y )} = ( x + wi / 2 , y + hi / 2 ) ,$$

where wi is the width, in pixels, of the material in each depth image, and hi is the length, in pixels, of the material in the same depth image.

In one embodiment, the object tracking module further uses a Kalman filter to predict moving path information of the material.

In one embodiment, the object tracking module further calculates with the position information and the moving path information and uses a Hungarian algorithm to determine the lowest path-matching cost C as follows:

$$C = [ \begin{matrix}{f}_{{s}_{1} , {r}_{1}} & {f}_{{s}_{1} , {r}_{2}} & … & {f}_{{s}_{1} , {r}_{n}} \\ {f}_{{s}_{2} , {r}_{1}} & {f}_{{s}_{2} , {r}_{2}} & … & {f}_{{s}_{2} , {r}_{n}} \\ ⋮ & ⋮ & ⋱ & ⋮ \\ {f}_{{s}_{m} , {r}_{1}} & {f}_{{s}_{m} , {r}_{2}} & … & {f}_{{s}_{m} , {r}_{n}}\end{matrix} ]$$

where f_{si,ri}=(zₛᵢ−Hxᵣᵢ)^{T}(R+HPH^{T})⁻¹(zₛᵢ−Hxᵣᵢ)+log|R+HPH^{T}|,
where z is a measured value (the detected centroid of the current object), H is a measurement matrix, x is a state matrix (the centroid predicted by the Kalman filter), R is measurement noise, and P is a covariance matrix.

In one embodiment, the material inspection system further includes a defect detection module. The defect detection module feeds each depth image into a defect identification model, which performs an algorithm to identify whether or not the material has any defect. When the material has a defect, the defect detection module calculates a defect ratio according to the following equation:

$$defect ratio = the number of pixels corresponding to the defect in the material in each depth image / the number of pixels corresponding to the material in the same depth image .$$

In one embodiment, the conveying portion includes: a driving unit that defines a circulation path between the first end and the second end; and at least two first shafts that are spaced apart from each other and arranged along the circulation path in a way that allows the material to be supported on the first shafts and be conveyed as the first shafts are driven to move along the circulation path. In addition, the driving portion is provided on one side of the first shafts to drive the first shafts into rotation and to simultaneously drive the material into rotation.

In one embodiment, the driving portion has a second shaft located between the first shafts and movable between a driving position and a far position. When at the driving position, the second shaft is adjacent to the first shafts, causes the first shafts to rotate in a synchronous manner, with the rotation direction of each first shaft being opposite to the rotation direction of the second shaft, and thereby drives the material on the first shafts into rotation. When at the far position, the second shaft is far from the first shafts.

In one embodiment, the driving unit includes: a conveyor belt arranged along the circulation path; and a power source connected to the conveyor belt in order to drive the conveyor belt into a circulating motion and thereby drive the first shafts and the second shaft, both provided on the conveyor belt, to move synchronously.

The present invention overcomes the limitations of the conventional volume calculation method, increases the degree of automation, prevents the risks of human or mechanical errors, and can therefore enhance production efficiency effectively.

### BRIEF DESCRIPTION OF DRAWINGS

FIG. 1 is a system block diagram of a preferred embodiment of the present invention.

FIG. 2 is a schematic drawing of a preferred embodiment of the present invention.

FIG. 3 is an enlarged view of portion A in FIG. 2.

FIG. 4 shows the parts in FIG. 3 in operation.

FIG. 5 is the flowchart of a preferred embodiment of the present invention.

FIG. 6(a), (b), and (c) are actual images showing how objects are tracked.

FIG. 7(a), (b), and (c) are actual images showing how objects are labeled and tracked.

FIG. 8(a), (b), and (c) are actual images showing object tilt correction.

FIG. 8(d) is an actual image showing how the length and width in pixels of an object are obtained.

FIG. 9 is actual images showing depth matrix fusion (also known as depth map fusion).

FIG. 10 is an actual image showing defect identification.

### DETAILED DESCRIPTION OF THE INVENTION

The “training models” disclosed in the present invention are constructed with machine learning and deep learning algorithms that are commonly used, such as YOLO (You Only Look Once), RCNN (Region-based Convolutional Neural Network), VGG (Visual Geometry Group), ResNet (Residual Network), and Inception (GoogleNet), and each trained model is suitable for a task such as target detection, image classification, or image recognition. In this embodiment, each training model is trained and tested with actual images, labeled data, and relevant parameters or data.

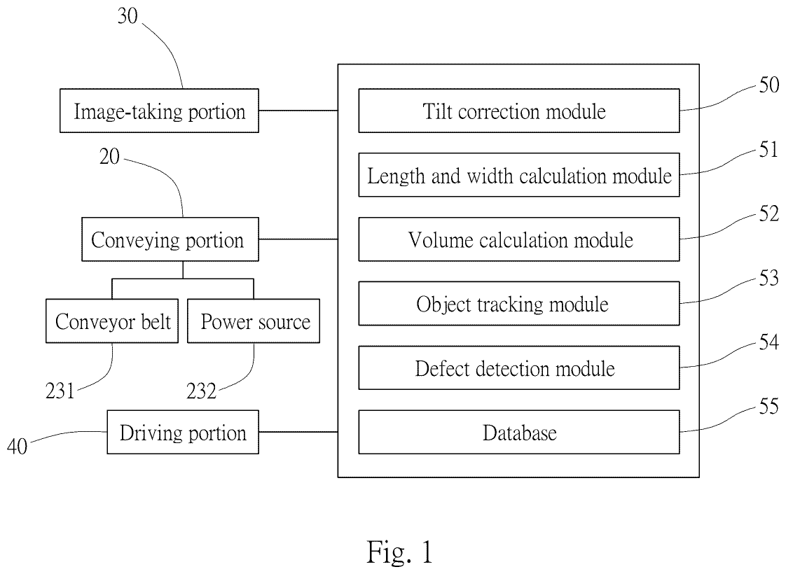

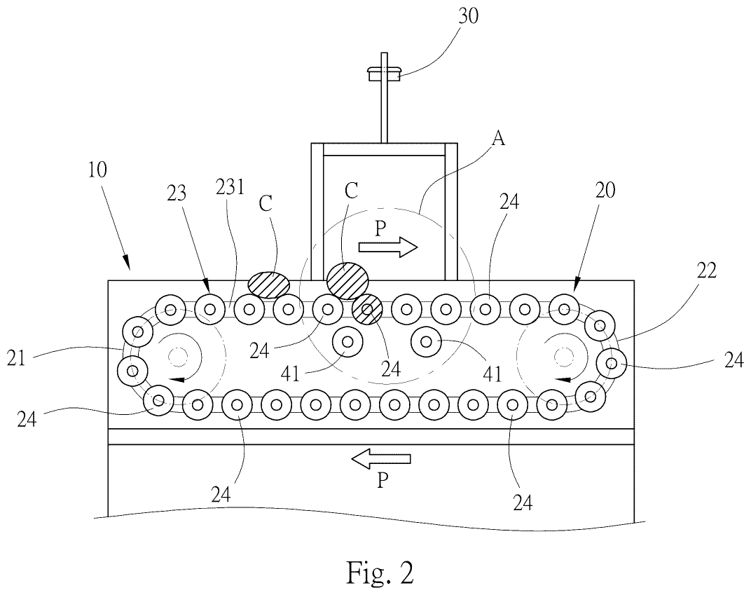

Referring to FIG. 1 and FIG. 2, the material inspection system according to a preferred embodiment of the present invention essentially includes a supporting structure 10, a conveying portion 20, an image-taking portion 30, a driving portion 40, a tilt correction module 50, a length and width calculation module 51, a volume calculation module 52, an object tracking module 53, a defect detection module 54, and a database 55. Each of the tilt correction module 50, the length and width calculation module 51, the volume calculation module 52, the object tracking module 53, and the defect detection module 54 is a program, a piece of software, or a training model preset for and run by a computing device and can perform such operations as “carrying out an algorithm,” “analyzing,” “judging,” “comparing,” “matching,” and “computing” on the data received. The computing device may be, but is not limited to, a personal computer (PC), a laptop computer, a tablet computer, a smartphone, or other apparatuses capable of computation or performing an algorithm. In this embodiment, the computing device is a Jetson Nano Kit, which is an embedded artificial intelligence (AI) computing device having a smaller volume than a computer and therefore not only meets the practical needs of use, but also contributes to reducing the volume of the entire system effectively. The embedded AI computing device may transmit/receive signals or commands to/from the image-taking portion 30, the driving portion 40, and the database 55 through any wired or wireless medium such as the fourth generation of cellular network technology (4G), the fifth generation of cellular network technology (5G), Wi-Fi, Bluetooth, Near-Field Communication (NFC), or Radio-Frequency Identification (RFID), either directly or indirectly.

The database 55 is a physical storage medium and may be, for example, a phase-change memory (PRAM), a static random-access memory (SRAM), a dynamic random-access memory (DRAM), a flash drive, a read-only memory (ROM), a random-access memory (RAM), a magnetic disk, or an optical disk. The database 55 is provided on the computing device to store the computation results of such modules as the tilt correction module 50, the length and width calculation module 51, the volume calculation module 52, the object tracking module 53, and the defect detection module 54 or the images taken by the image-taking portion 30.

The supporting structure 10 is a physical foundation to which the other components are attached. The conveying portion 20 is provided on the supporting structure 10, has a first end 21 and a second end 22, and is configured to convey a material C from the first end 21 to the second end 22. The material C is farm produce such as a white radish, a carrot, a potato, a cucumber, an apple, or an orange, and there is no limitation on the size or shape of the material C. The material C may have a spherical shape, a conical shape, a columnar shape, or other geometric shapes. The conveying portion 20 includes: a driving unit 23 that defines a circulation path P between the first end 21 and the second end 22; and at least two first shafts 24 that are arranged at intervals along the circulation path P in a way that allows the material C to be supported on the first shafts 24 and be conveyed as the first shafts 24 are driven to move along the circulation path P. As the material inspection system is a customized product, the spacing between the first shafts 24 and the size of the supporting structure 10 can be adjusted according to the size of the material C or as needed.

More specifically, the driving unit 23 includes a conveyor belt 231 and a power source 232. The conveyor belt 231 is arranged along the circulation path P. The power source 232 is connected to the conveyor belt 231 in order to drive the conveyor belt 231 to move in a circulating manner, thereby driving the first shafts 24, which are provided on the conveyor belt 231, to move synchronously. The power source 232 is a power device such as a motor.

The image-taking portion 30 has an imaging lens, is mounted on the supporting structure 10, and corresponds to the conveying portion 20 such that the field of view of the imaging lens encompasses at least a portion, or the entirety, of the path along which the material C is moved on the conveying portion 20, thereby allowing the image-taking portion 30 to take images of the material C.

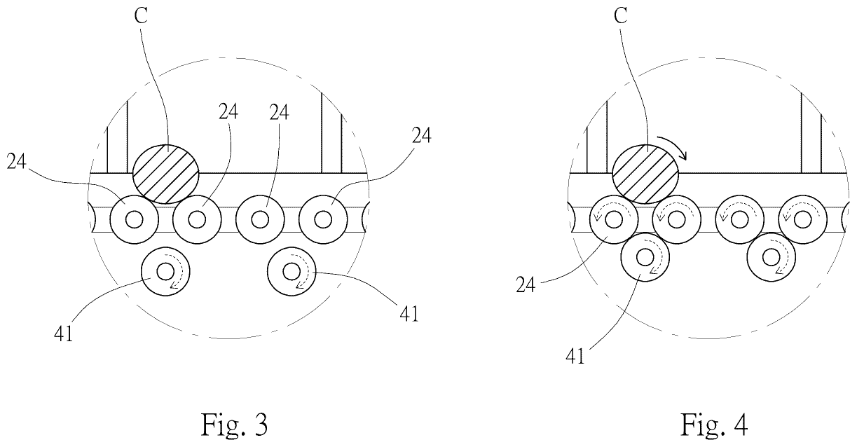

The driving portion 40 is located on the conveying portion 20 and is configured to drive the material C into rotation such that the portion of the material C that faces the image-taking portion 30 is changed at different time points, i.e., different portions of the material C face the image-taking portion 30 at different time points. This allows the image-taking portion 30 to take a plurality of depth images of the different portions of the material C while the material C is rotating. More specifically, the driving portion 40 has a second shaft 41, and the second shaft 41 is located between the first shafts 24 and can be moved between a driving position and a far position. When at the driving position as shown in FIG. 4, the second shaft 41 is adjacent to the first shafts 24 and causes the first shafts 24 to rotate synchronously, with the rotation direction of each first shaft 24 being opposite to the rotation direction of the second shaft 41, and in doing so, the second shaft 41 also drives the material C on the first shafts 24 to rotate so that the image-taking portion 30 can take images of the material C comprehensively from all angles and thereby obtain information about the material C from multiple angles. When at the far position as shown in FIG. 3, the second shaft 41 is far from the first shafts 24. In other words, the driving portion 40 can work in a first mode or a second mode. The first mode is a mode in which the second shaft 41 is at the far position, and in which the conveying portion 20 only conveys the material C forward. When it is detected that the material C enters the field of view of the imaging lens, the driving portion 40 is switched from the first mode to the second mode, in which the second shaft 41 is at the driving position to drive the material C into rotation.

The movement of the second shaft 41 between the driving position and the far position can be achieved with a lifting and lowering mechanism. The lifting and lowering mechanism may be, but is not limited to, a pneumatic, hydraulic, or electric mechanism, all of which are equally capable of serving the purpose of adjusting or changing the second shaft 41 in height.

In addition, the second shaft 41 in this embodiment is driven by the conveyor belt 231 to move along the circulation path P. In other feasible embodiments, the driving portion 40 may be fixed at a predetermined position instead.

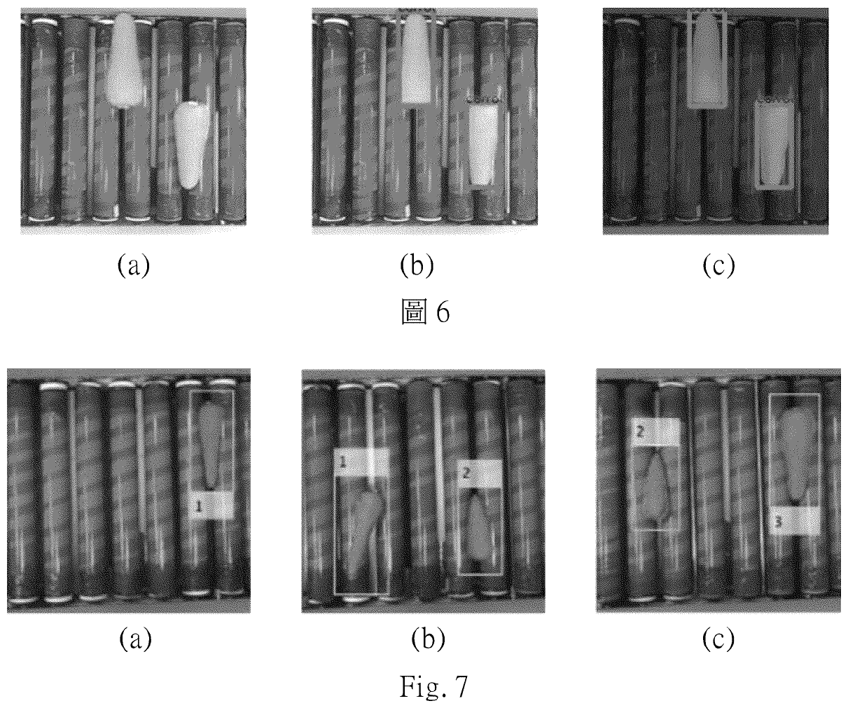

The object tracking module 53 identifies the material C in each image taken by the image-taking portion 30, obtains position information of the material C through analysis, and marks each image with a tracking label at the position where the material C is identified, as shown in FIG. 7. The position information is obtained by determining a centroid according to the following equation, and the centroid is used as a basis for tracking:

$${centroid}_{( x , y )} = ( x + wi / 2 , y + hi / 2 ) ,$$

where wi and hi are the width and length, both in pixels, of the material in each depth image, respectively.

When there are a plurality of materials C, the object tracking module 53 can track the materials individually at the same time, and this helps increase the production efficiency of the materials to the greatest extent. By contrast, the conventional identification techniques can identify only one object at a time and therefore has very low efficiency.

The object tracking module 53 further uses a Kalman filter to predict moving path information of the material C, wherein the calculation equations of the Kalman filter include the following:

$$\begin{matrix}x = {[ {p}_{x} , {p}_{y} , {v}_{x} , {v}_{y} ]}^{T} , \\ F = [ \begin{matrix}1 & 0 & δ t & 0 \\ 0 & 1 & 0 & δ t \\ 0 & 0 & 1 & 0 \\ 0 & 0 & 0 & 1\end{matrix} ] , \\ H = [ \begin{matrix}1 & 0 & 0 & 0 \\ 0 & 1 & 0 & 0\end{matrix} ] , \\ {\hat{x}}_{{t}^{-}} = F {\hat{x}}_{t - 1} + {η}_{t - 1} , \\ {z}_{t} = {Hx}_{t} + {ζ}_{t} , \\ {P}_{{t}^{-}} = {FP}_{t - 1} {F}^{T} + Q , \\ {K}_{t} = {P}_{{t}^{-}} {{H}^{T} ( {HP}_{{t}^{-}} {H}^{T} + R )}^{- 1} , \\ {\hat{x}}_{t} = {\hat{x}}_{{t}^{-}} + {K}_{t} ( {z}_{t} - H {\hat{x}}_{{t}^{-}} ) , and \\ {P}_{t} = ( I ‐ {K}_{t} H ) + {P}_{{t}^{-}} ,\end{matrix}$$

where pₓ and p_{y} are centroid positions, vₓ and v_{y} are moving speeds along the x and y axes respectively, F and H are a state transition matrix and a measurement matrix respectively, ηₜ and ζₜ are Gaussian noise with error covariances Q and R respectively, \hat{x}ₜ—is a priori-state estimate, \hat{x}ₜ is a posteriori-state estimate, Pₜ—is a priori-state covariance matrix, Pₜ is a posteriori-state covariance matrix, and Kₜ is the Kalman gain.

The object tracking module 53 further calculates with the position information and the moving path information and uses a Hungarian algorithm to determine the lowest path-matching cost C as follows:

$$C = [ \begin{matrix}{f}_{{s}_{1} , {r}_{1}} & {f}_{{s}_{1} , {r}_{2}} & … & {f}_{{s}_{1} , {r}_{n}} \\ {f}_{{s}_{2} , {r}_{1}} & {f}_{{s}_{2} , {r}_{2}} & … & {f}_{{s}_{2} , {r}_{n}} \\ ⋮ & ⋮ & ⋱ & ⋮ \\ {f}_{{s}_{m} , {r}_{1}} & {f}_{{s}_{m} , {r}_{2}} & … & {f}_{{s}_{m} , {r}_{n}}\end{matrix} ]$$

where f_{si,ri}=(zₛᵢ−Hxᵣᵢ)^{T}(R+HPH^{T})⁻¹(zₛᵢ−HXᵣᵢ)+log|R+HPH^{T}|,
where z is a measured value (the detected centroid of the current object), H is a measurement matrix, x is a state matrix (the centroid predicted by the Kalman filter), R is measurement noise, and P is a covariance matrix.

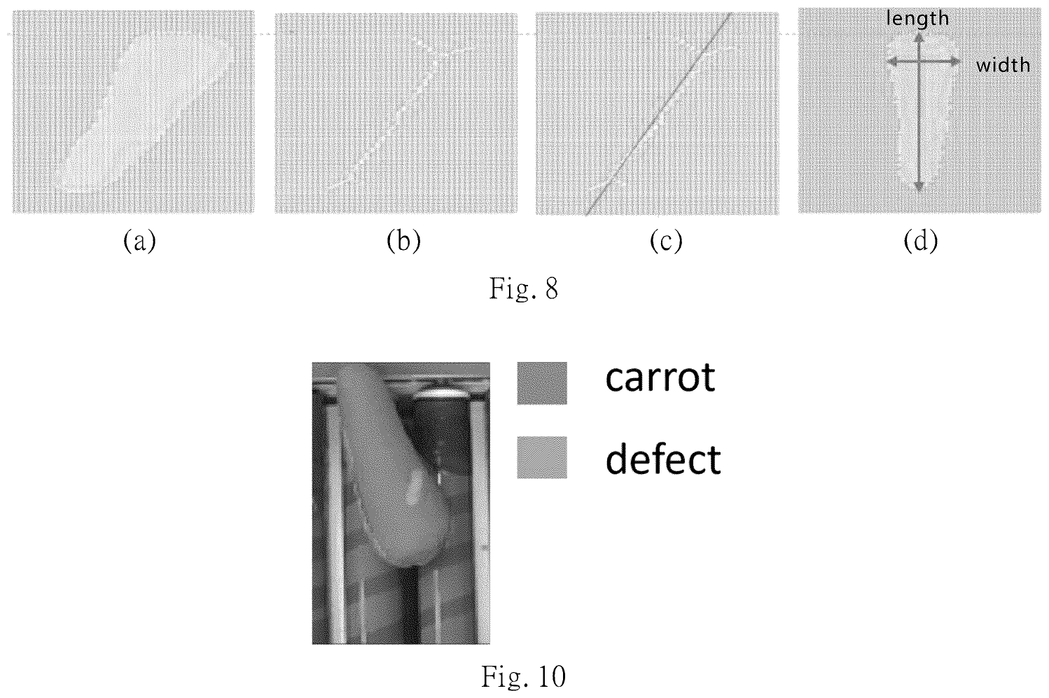

The tilt correction module 50 obtains the position information of the material C from the object tracking module 53, then uses an erosion algorithm to extract the skeleton of the object (i.e., the material C), obtains the central axis of the material C by curve fitting, takes the slope of the central axis as the tilt angle of the material C, and corrects the tilt angle by image rotation, as shown in FIG. 8(a), (b), and (c). In practice, the material C, e.g., a carrot, may have an irregular shape and therefore have problem staying perpendicular to the imaging lens while on the first shafts 24. The tilt correction module 50 can correct images taken of the material C while the material C is rolling or bouncing, thereby allowing the correct data to be calculated.

Aside from the tilt correction, the images or depth images may be further subjected to other pre-processing operations. Simply put, image pre-processing refers to a series of operations performed on an image before the image is analyzed. For example, image pre-processing includes adjustments in contrast and brightness, grayscale conversion, smoothing, edge detection, binarization, cropping and scaling, geometric transformation, color space conversion, morphological operations, and noise reduction. The pre-processing operations prepare for subsequent analyses and applications by assisting in image quality improvement, the extraction of useful information, noise reduction, and feature enhancement.

The length and width calculation module 51 uses the images having received tilt correction, takes the length (in pixels) and width (in pixels) of the material C in each such image as inputs (see FIG. 8(d)), and calculates the length (in cm) and width (in cm) of the material C through a width and length training model. In contrast to converting a distance in an image to the actual distance using a conventional method that requires the camera position, camera parameters, and actual roller size to be obtained and projective transformation to be performed on the image, the present embodiment uses only the width and length training model for calculation. The width and length training model can train and learn by itself, without having to conduct the various steps or parameter adjustment in the conventional method, and when the camera position or the conveying platform is changed, there is no need to reset complicated parameters or perform additional steps, either.

The volume calculation module 52 extracts the depth information in each depth image and feeds each piece of depth information into a deep learning model in order to calculate the volume of the material C.

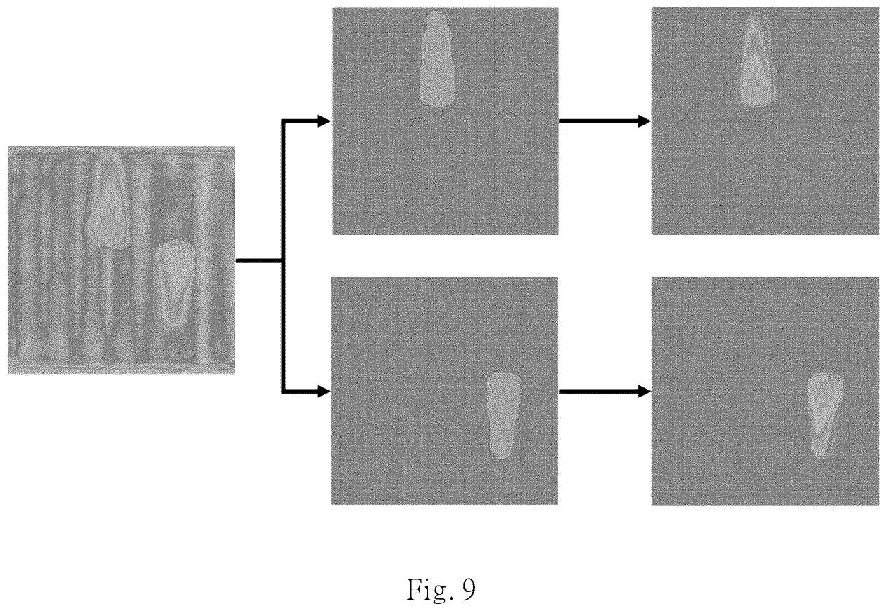

The volume calculation module 52 further performs the step of depth matrix fusion (or depth map fusion) by analyzing each piece of depth information to produce the corresponding depth matrix, multiplying each depth matrix by the position information pixel-wise to obtain a plurality of corresponding depth values, and determining a material position and a non-material position in each piece of depth information according to the corresponding depth values. The volume calculation module 52 then excludes the portion of each piece of depth information that is determined to be a non-material position, and performs volume calculation based on the portion of each piece of depth information that is determined to be a material position. Each depth value is 0 or 1. The depth value 1 indicates that the corresponding portion of a piece of depth information is a material position, while the depth value 0 indicates that the corresponding portion of a piece of depth information is a non-material position. With the depth values serving to eliminate the non-material positions, the purity of volume prediction is preserved, as shown in FIG. 9.

The defect detection module 54 feeds each depth image into a defect identification model (e.g., a semantic segmentation model for defect identification) in order for the model to perform an algorithm and thereby identify whether or not the material C has any defect, as shown in FIG. 10. When the material C has a defect, the defect detection module 54 calculates a defect ratio according to the following equation:

defect ratio=the number of pixels corresponding to the defect in the material in each depth image/the number of pixels corresponding to the material in the same depth image.

Thus, the defect detection module 54 analyzes defects in a quantitative manner. A standard value can be further used as a basis for grading (e.g., the defect ratio exceeds 10%) to improve the subjective bias common in conventional manual inspection. The quantified data can also be stored in the database 55 or further analyzed.

In addition, the aforesaid tracking labels and the identification result of the defect detection module 54 enable comprehensive tracking of the material C. Take farm produce for example. Individual identification (ID) information can be stablished to distinguish produce of different origins and to create a produce database that records annual yields, produce damage levels, and produce sizes.

Based on the foregoing description, a specific mode of implementing the present invention is as follows.

To begin with, referring to FIG. 1, the material C is supported on two adjacent first shafts 24 and is moved along with the first shafts 24 from the first end 21 to the second end 22 along the circulation path P.

When the material C moves into the field of view of the imaging lens, the image-taking portion 30 takes images of the material C, and the object tracking module 53 tracks the material C based on the images. It is worth mentioning that when there are a plurality of materials C, the object tracking module 53 can track the multiple objects (i.e., materials C) at the same time in order for the volumes of the multiple objects to be calculated simultaneously. If a conventional technique is used, it will be necessary to construct 3D models for the multiple objects separately, and this will prolong the computation time, making it impossible to show the computation results in real time or rapidly.

The second shaft 41 is then driven to move from the far position to the driving position, as shown in FIG. 4. Once the second shaft 41 is in contact with the first shafts 24, the first shafts 24 are driven to rotate synchronously, with the rotation direction of each first shaft 24 being opposite to that of the second shaft 41. Consequently, the material C, which is on the first shafts 24, is driven to rotate, allowing the image-taking portion 30 to take a plurality of depth images of different portions of the material C while the material C is rotating.

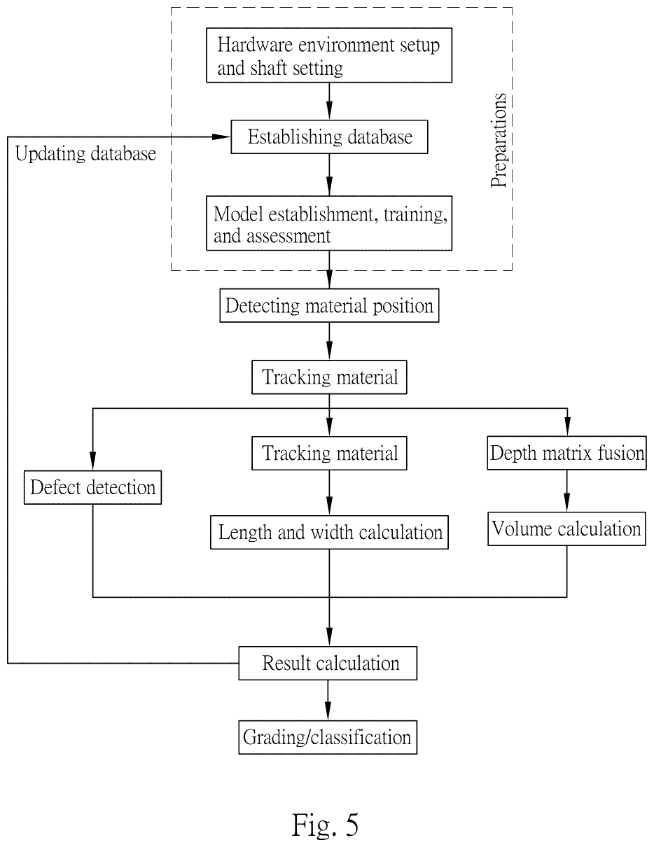

Lastly, the object tracking module 53, the tilt correction module 50, the length and width calculation module 51, the volume calculation module 52, and the defect detection module 54 perform their respective calculations or analyses, i.e., conduct material tracking, image angle correction, calculation of the length and width of the material, calculation of the volume of the material, and defect detection, respectively, as shown in the flowchart of FIG. 5. The calculation and analysis results are sent to the database 55 to update the data therein so that by continually receiving the data fed back, the database 55 can be established as a constantly updated dynamic database that can fine-tune the training models persistently, thereby allowing the accuracy of the models to increase with time. Moreover, the data stored in the database 55 can be provided to various other platforms for the purpose of resource sharing. For example, the quantified data can be provided to other users in the upstream industries or recorded in conjunction with climate changes to facilitate analysis of the effects of climate changes on farm produce.

What is claimed is:

1. A material inspection system, comprising:
a supporting structure;
a conveying portion provided on the supporting structure and having a first end and a second end, the conveying portion being configured to convey a material from the first end to the second end;
an image-taking portion having an imaging lens, wherein the imaging lens has a field of view encompassing at least a portion of a path along which the material is moved on the conveying portion, in order for the image-taking portion to take images of the material;
a driving portion located on the conveying portion and configured to drive the material into rotation such that different portions of the material face the image-taking portion at different time points to allow the image-taking portion to take a plurality of depth images of the different portions of the material while the material is in rotation;
a volume calculation module for extracting depth information in each said depth image and feeding each piece of said depth information into a deep learning model to calculate a volume of the material; and
an object tracking module for identifying the material in each said image taken by the image-taking portion, obtaining position information of the material through analysis, and marking each said image with a tracking label at a position where the material is identified.

2. The material inspection system of claim 1, wherein the volume calculation module further analyzes each piece of said depth information to produce a corresponding depth matrix, multiplies each said depth matrix by the position information pixel-wise to obtain a plurality of corresponding depth values, determines a material position and a non-material position in each piece of said depth information according to corresponding said depth values, and performs volume calculation based on portions of the depth information that are determined to be said material positions.

3. The material inspection system of claim 2, wherein each said depth value is 0 or 1, with 1 indicating that a corresponding portion of a piece of said depth information is a said material position, and 0 indicating that a corresponding portion of a piece of said depth information is a said non-material position.

4. The material inspection system of claim 1, wherein there are a plurality of said materials, and the object tracking module is able to track the materials individually in a simultaneous manner.

5. The material inspection system of claim 1, wherein the position information is obtained by determining a centroid according to the following equation, and the centroid is used as a basis for tracking:
$${centroid}_{( x , y )} = ( x + wi / 2 , y + hi / 2 ) ,$$
where wi and hi are a width and a length, both in pixels, of the material in each said depth image, respectively.

6. The material inspection system of claim 1, wherein the object tracking module further uses a Kalman filter to predict moving path information of the material.

7. The material inspection system of claim 6, wherein the object tracking module further calculates with the position information and the moving path information and uses a Hungarian algorithm to determine a lowest path-matching cost C as follows:
$$C = [ \begin{matrix}{f}_{{s}_{1} , {r}_{1}} & {f}_{{s}_{1} , {r}_{2}} & … & {f}_{{s}_{1} , {r}_{n}} \\ {f}_{{s}_{2} , {r}_{1}} & {f}_{{s}_{2} , {r}_{2}} & … & {f}_{{s}_{2} , {r}_{n}} \\ ⋮ & ⋮ & ⋱ & ⋮ \\ {f}_{{s}_{m} , {r}_{1}} & {f}_{{s}_{m} , {r}_{2}} & … & {f}_{{s}_{m} , {r}_{n}}\end{matrix} ]$$
where f_{si,ri}=(zₛᵢ−HXᵣᵢ)^{T}(R+HPH^{T})⁻¹(zₛᵢ−HXᵣᵢ)+log|R+HPH^{T}|,
where z is a measured value, H is a measurement matrix, x is a state matrix, R is measurement noise, and P is a covariance matrix.

8. The material inspection system of claim 1, further comprising a defect detection module, wherein the defect detection module feeds each said depth image into a defect identification model in order for the defect identification model to perform an algorithm to identify whether or not the material has any defect, and when the material has a defect, the defect detection module calculates a defect ratio for any said depth image according to the following equation:
$$defect ratio = the number of pixels corresponding to the defect in the material in the depth image / the number of pixels corresponding to the material in the depth image .$$

9. The material inspection system of claim 1, wherein the conveying portion comprises:
a driving unit defining a circulation path between the first end and the second end; and
at least two first shafts spaced apart from each other and arranged along the circulation path in a way that allows the material to be supported on the first shafts and be conveyed as the first shafts are driven to move along the circulation path;
wherein the driving portion is provided on one side of the first shafts and is configured to drive the first shafts into rotation and thereby drive the material into rotation simultaneously.

10. The material inspection system of claim 9, wherein the driving portion has a second shaft located between the first shafts and movable between a driving position and a far position; when at the driving position, the second shaft is adjacent to the first shafts, causes the first shafts to rotate synchronously, with a rotation direction of each said first shaft being opposite to a rotation direction of the second shaft, and thereby drives the material on the first shafts into rotation; and when at the far position, the second shaft is far from the first shafts.

11. The material inspection system of claim 10, wherein the driving unit comprises:
a conveyor belt arranged along the circulation path; and
a power source connected to the conveyor belt to drive the conveyor belt into a circulating motion and thereby drive the first shafts and the second shaft, both provided on the conveyor belt, to move synchronously.
